(19) United States
(12) Patent Application Publication
(10) Pub. No.: US 2026/0142038 A1
(43) Pub. Date: May 21, 2026

(54) TYPE II DIABETES PREDICTION SYSTEM AND METHOD

(71) Applicant: Cigna Intellectual Property, Inc., Wilmington, DE (US)

(72) Inventor: Angela Tollis, Canton, CT (US)

(21) Appl. No.: 19/389,524

(22) Filed: Nov. 14, 2025

Related U.S. Application Data
(60) Provisional application No. 63/720,909, filed on Nov. 15, 2024.

(51) Int. Cl. G16H 50/30; G16H 10/60

(52) U.S. Cl. CPC G16H 50/30; G16H 10/60

(57) ABSTRACT

Systems and methods for predicting a likelihood of a patient developing Type II diabetes are provided. Behavioral health data, mental health data, and/or neurological health data is obtained, risk factors from the health data are identified, and an at-risk score is calculated based on the one or more risk factors. The at-risk score is indicative of the likelihood of the patient developing Type II diabetes. The different types of health data can be weighed differently in calculating the at-risk score.

### CROSS-REFERENCE TO RELATED APPLICATIONS

This application claims priority to U.S. Provisional Application No. 63/720,909 (filed 15 Nov. 2024), the entire disclosure of which is incorporated herein by reference.

### BACKGROUND

Diagnoses of Type II diabetes is growing in prevalence. This condition can pose significant health risks, progressing into more serious chronic illnesses like kidney disease. If the propensity for developing Type II diabetes is identified early, before the presentation of physiological symptoms, preventive interventions can be supplied, delaying or avoiding the onset of Type II diabetes.

Pre-diabetic patients may not understand from healthcare providers that the development of Type II diabetes can be avoided through diet, exercise, and lifestyle modifications. Currently, healthcare industries mostly address Type II diabetes after the patient presents high A1C levels and has progressed to requiring medication to treat the Type II diabetes. For example, while estimates show that ninety-six millions adults in the United States are pre-diabetic (and leading toward Type II diabetes and potentially Type I diabetes), more than eighty percent of these adults may be unaware of their condition.

Certain factors increase the risk of developing Type II diabetes, including diet and exercise. But other factors also may increase this risk. A need may exist for considering additional factors to provide for early identification of adults with increased risk for developing Type II diabetes to intervene early and often. This can help these adults avoid developing Type II or Type I diabetes.

### BRIEF SUMMARY

In one example, a method for predicting a likelihood of a patient developing Type II diabetes is provided. The method can include obtaining one or more of behavioral health data, mental health data, or neurological health data of the patient, identifying one or more risk factors from the one or more of the behavioral health data, the mental health data, or the neurological health data, and calculating an at-risk score based on the one or more risk factors, the at-risk score indicative of the likelihood of the patient developing Type II diabetes. The method also can include implementing one or more intervention actions responsive to the at-risk score exceeding a designated threshold. The one or more intervention actions can include one or more of scheduling a visit with a healthcare provider, delivering meals to the patient, scheduling exercise or training sessions with the patient, scheduling mental health counseling sessions with the patient, or prescribing one or more medications to the patient. The behavioral health data can be obtained and includes one or more of the patient consuming alcohol, the patient having obstructive sleep apnea, the patient consuming a low-fiber diet with a high glycemic index, the patient consuming processed meats and insufficient non-processed meats, or the patient consuming soda. The mental health data can be obtained and include one or more of the patient experiencing childhood abuse, the patient having post-traumatic stress disorder, the patient experiencing a traumatic event, the patient having excess stress, or the patient having a depression diagnosis. The neurological health data can be obtained and include one or more of the patient having a spinal cord injury, or the patient having an inflammatory process. The method also can include obtaining physical health data, and the one or more risk factors also can be identified from the physical health data. The physical health data can include one or more of a family history of diabetes, the patient being obese, the patient having a sedentary lifestyle, the patient having high blood pressure, or the patient being a smoker. The at-risk score can be calculated based on multiple ones of the one or more risk factors. The at-risk score can be calculated by weighing the multiple ones of the one or more risk factors differently. The risk factors from the behavioral health data can be weighed more heavily than the risk factors from the mental health data or from the neurological health data in calculating the at-risk score. The risk factors from the physical health data can be weighed more heavily than the risk factors from the mental health data or from the neurological health data in calculating the at-risk score.

In another example, an ASIC for an ANN is provided. The ASIC can include neurons organized in an array, with each of the neurons including a register, a processing element, and at least one input. The ASIC also can include synaptic circuits, with each of the synaptic circuits including a memory for storing a synaptic weight. Each of the neurons can be connected to at least one other of the neurons via at least one of the synaptic circuits. The processing elements of the neurons can obtain one or more of behavioral health data, mental health data, or neurological health data of a patient, identify one or more risk factors from the one or more of the behavioral health data, the mental health data, or the neurological health data, and calculate an at-risk score based on the one or more risk factors, the at-risk score indicative of a likelihood of the patient developing Type II diabetes. The processing elements of the neurons can implement one or more intervention actions responsive to the at-risk score exceeding a designated threshold. The one or more intervention actions can include one or more of scheduling a visit with a healthcare provider, delivering meals to the patient, scheduling exercise or training sessions with the patient, scheduling mental health counseling sessions with the patient, or prescribing one or more medications to the patient. The processing elements of the neurons can obtain the behavioral health data that includes one or more of the patient consuming alcohol, the patient having obstructive sleep apnea, the patient consuming a low-fiber diet with a high glycemic index, the patient consuming processed meats and insufficient non-processed meats, or the patient consuming soda. The processing elements of the neurons can obtain the mental health data that includes one or more of the patient experiencing childhood abuse, the patient having post-traumatic stress disorder, the patient experiencing a traumatic event, the patient having excess stress, or the patient having a depression diagnosis. The processing elements of the neurons can obtain the neurological health data that includes one or more of the patient having a spinal cord injury, or the patient having an inflammatory process.

In another example, a Type II diabetes prediction system is provided. The prediction system can include a control unit that can obtain one or more of behavioral health data, mental health data, or neurological health data of the patient. The control unit can identify one or more risk factors from the one or more of the behavioral health data, the mental health data, or the neurological health data. The control unit can calculate an at-risk score based on the one or more risk factors. The at-risk score can indicate the likelihood of the patient developing Type II diabetes. The control unit can output the likelihood of the patient developing Type II diabetes to an intervention system that implements one or more intervention actions responsive to the at-risk score exceeding a designated threshold. The one or more intervention actions can include one or more of scheduling a visit with a healthcare provider, delivering meals to the patient, scheduling exercise or training sessions with the patient, scheduling mental health counseling sessions with the patient, or prescribing one or more medications to the patient. The control unit can obtain the behavioral health data that includes one or more of the patient consuming alcohol, the patient having obstructive sleep apnea, the patient consuming a low-fiber diet with a high glycemic index, the patient consuming processed meats and insufficient non-processed meats, or the patient consuming soda. The control unit can obtain the mental health data that includes one or more of the patient experiencing childhood abuse, the patient having post-traumatic stress disorder, the patient experiencing a traumatic event, the patient having excess stress, or the patient having a depression diagnosis. The control unit can obtain physical health data and identify the one or more risk factors using the physical health data. The control unit can calculate the at-risk score based on multiple ones of the one or more risk factors. The control unit can calculate the at-risk score by weighing the multiple ones of the one or more risk factors differently. The risk factors from the behavioral health data can be weighed more heavily than the risk factors from the mental health data or from the neurological health data in calculating the at-risk score. The risk factors from the physical health data can be weighed more heavily than the risk factors from the mental health data or from the neurological health data in calculating the at-risk score.

### BRIEF DESCRIPTION OF THE SEVERAL VIEWS OF THE DRAWINGS

In the drawings, which are not necessarily drawn to scale, like numerals may describe similar components in different views. To easily identify the discussion of any particular element or act, the most significant digit or digits in a reference number refer to the figure number in which that element is first introduced. Some embodiments are illustrated by way of example, and not limitation, in the figures of the accompanying drawings in which:

FIG. 1 illustrates one example of a Type II diabetes prediction system;

FIG. 2 illustrates a flowchart of a method for predicting a risk or likelihood that a patient will develop Type II diabetes;

FIG. 3 illustrates one example of a machine learning (ML)/artificial intelligence (AI) system; and

FIG. 4 illustrates one example of a control unit shown in FIG. 1 embodied in at least one ASIC.

### DETAILED DESCRIPTION

The description that follows includes systems, methods, techniques, instruction sequences, and computing machine program products that embody illustrative embodiments of the disclosure. In the following description, for the purposes of explanation, numerous specific details are set forth in order to provide an understanding of various embodiments of the inventive subject matter. It will be evident, however, to those skilled in the art, that embodiments of the inventive subject matter may be practiced without these specific details. In general, well-known instruction instances, protocols, structures, and techniques are not necessarily shown in detail.

Examples of the inventive subject matter described herein provide for inventive Type II diabetes prediction systems and methods that can more accurately predict development of Type II diabetes than some known or conventional algorithms due to the inventive systems and methods considering whole-person-health criteria, including socio-behavioral, mental, and/or neurological risk factors. While some known predictive models may rely solely on physical risk factors like age, body mass index, race, hemoglobin A1C levels, and/or fasting plasma glucose levels, these known predictive models do not consider mental health, and do not take a person's mental health and neurological conditions, and/or behavioral conditions into account.

Not all embodiments of the inventive subject matter described herein may take into account mental health and neurological conditions and behavioral conditions into account when predicting the risk or likelihood of a patient developing Type II diabetes. For example, some of these factors may be unavailable or not quantifiable. Accordingly, unless expressly otherwise stated, the inventive subject matter may take any combination of the mental health factors, neurological conditions, and/or behavioral conditions when predicting the risk or likelihood of a patient developing Type II diabetes.

The systems and methods can use a trained model to predict, based on a specific set of criteria, whether an adult is at risk of developing diabetes. The predictive model can use decision tree logic and machine learning techniques to focus on more influential criteria and risk factors in predicting Type II diabetes. When used with patients early in their physical health journey with their healthcare provider(s), the systems and methods can use the model to surface or reveal evidence that a particular patient is at greater risk for developing diabetes. This can enable the healthcare provider(s) to develop pathways to a healthier future via preventive clinical interventions. The model can attribute separate and specific scores and weights to physical criteria, behavioral criteria, and mental/neurological criteria, and then combine the weighted scores to identify the risk or likelihood of the patients developing diabetes (e.g., Type II diabetes).

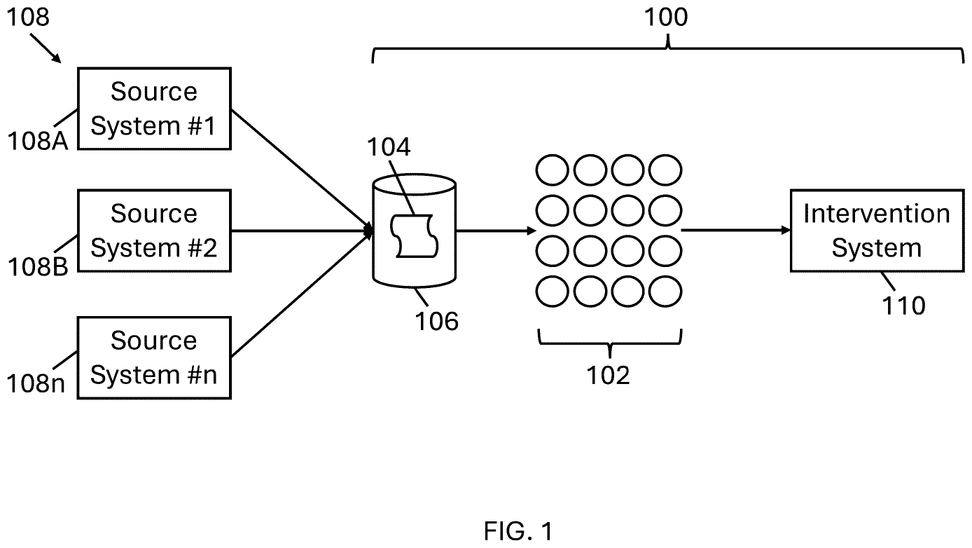

FIG. 1 illustrates one example of a Type II diabetes prediction system 100. The prediction system 100 can include a control unit 102, which can represent an artificial intelligence or machine learning system as described herein. The control unit 102 can be embodied in one or more processors (e.g., one or more application-specific integrated circuits, or ASICs; other integrated circuits; microprocessors; field programmable gate arrays; or the like) that perform the operations described in connection with the control unit 102. The control unit 102 can train, re-train or refine, and use a predictive model 104 stored in a tangible and non-transitory computer-readable storage medium, or computer memory 106 (e.g., one or more computer hard drives), to predict the risk or likelihood that a patient will develop Type II diabetes.

The predictive model 104 uniquely combines specific physical, mental, neurological, and/or behavioral risk factors in a demographic to determine whether a person is at risk of developing Type II diabetes. The control unit 102 can obtain (for storage in the memory 106 and inclusion in the model 104) population health lifestyle data, medical data, and criteria from evidence-based studies that contribute to and/or cause Type 2 diabetes. This data can be received from source systems 108 (e.g., source systems 108A, 108B, 108n). While three source systems 108 are shown, optionally, the data can be received from fewer or more source systems 108.

The source systems 108 can represent other tangible and non-transitory computer-readable storage media, computer systems, etc. that generate and communicate the data used by the predictive model 104. In one example, at least some of the source systems 108 are hospital systems, medical libraries, online article sources, or the like, which provide medical studies on populations of patients regarding factors leading to development of Type II diabetes. Examples include demographic data tracking systems such as WISEVOTER (wisevoter.com), benefit plan systems administrators running health reimbursement arrangements, publications such as PHYSCHOLOGY TODAY, the National Institute of Health, and the like. Other source systems 108 can be healthcare providers (e.g., providing clinical notes, test results, etc.), psychologists, healthcare benefit providers (e.g., providing claim data), wearable sensors (e.g., APPLE WATCH, FITBIT devices, WHOOP devices, or the like.

The data can include health data such as physical health data, mental health data, neurological health data, and/or behavioral health data. The physical health data can include information that describes or reveals a patient's past, present, or future physical health. Examples of physical health data include family histories of diseases including diabetes, obesity, indicators or presence of a sedentary lifestyle or physical inactivity, blood pressure measurements, whether the patient smokes and how much the patient smokes, etc. Additional examples of physical health data include biological data (e.g., genetic information or other biological markers), medical data (e.g., health conditions, diagnoses, treatments, medications, allergies, or medical histories), health measurements (e.g., data collected from wearable devices or other health monitoring tools, such as heart rate, blood pressure, sleep patterns, or activity levels), lifestyle data (e.g., information about diet, exercise habits, smoking status, or alcohol consumption), etc.

The mental health data can include information that describes or reveals the mental state, emotional well-being, and/or cognitive functioning of one or more patients. Examples of mental health data can include the presence of childhood abuse (regardless of other demographic factors or with particular demographics, such as Caucasian males), the experience of traumatic events, diagnoses of post-traumatic stress disorder, the presence of excess stress and inflammatory processes, diagnoses of depression, etc. Additional examples of mental health data include diagnostic data, such as mental health diagnoses of conditions like anxiety, bipolar disorder, or schizophrenia. The mental health data can include medication histories (e.g., prescribed medications, dosages, and treatment durations), therapy records (e.g., clinical notes and documentation from therapy sessions, such as treatment plans, progress notes, and goals), hospitalization records (e.g., information obtained during inpatient or outpatient treatment, such as treatment dates, diagnoses, and interventions), self-reported data (e.g., self-assessment tools used to measure symptoms of mental health conditions, mood tracking apps, online forums and social media, etc.), physiological data (e.g., brain imaging such as data from techniques like MRI or fMRI that can reveal structural or functional abnormalities in the brain, neuropsychological testing, etc.), and/or genetic data (e.g., information about genetic variations that may contribute to mental health conditions).

The neurological health data can include information related to the structure and function of the nervous system, including the brain, spinal cord, and nerves, as well as injuries (e.g., spinal cord injuries), disorders, diseases, medication, etc., that impact the nervous system. The neurological health data can be obtained from medical records (e.g., medical histories, symptoms, diagnoses, treatments, and medications related to neurological conditions), brain imaging, genetic information (e.g., specific genes that may increase the risk of neurological disorders), results of neuropsychological tests, wearable device data (e.g., data on sleep patterns, heart rate variability, and other physiological measures relevant to neurological health), etc.

The behavioral health data can include information about a patient's behaviors, such as the amount of alcohol consumption, the presence of obstructive sleep apnea, unhealthy diets (e.g., low fiber diets with high glycemic indices), consumption of processed meats (with or without the consumption of other meats), amounts of soda or soft drink consumption, etc.

One or more of the different types of physical health data, mental health data, neurological health data, and/or behavioral health data described above may be obtained for the patient, for the family of the patient, or for both the patient and/or family. For example, some health data from family members of the patient may be helpful in determining whether the patient is at increased risk or likelihood for developing Type II diabetes. This health data can be obtained for immediate family members (e.g., family members of the patient via blood connection for a single generation, such as the patient's parents, brother(s), and/or sister(s) by birth), extended family members (e.g., family members of the patient via blood connection for two or more generations, such as the first generation described above and one or more grandparents, aunts, uncles, cousins, etc.), or combination of single and multi-generational family members.

The control unit 102 of the prediction system 100 can use the predictive model 104 to examine the mental health data, neurological health data, and/or behavioral health data of patients (along with or separate from the physical health data) of patients to predict the risk or likelihood of the patients developing Type II diabetes. As described herein, the model 104 can be trained and refined using clinical studies, medical articles, medical histories of other patients known to have developed Type II diabetes, medical histories of other patients known to have not developed Type II diabetes, etc. The control unit 102 can examine this information to predict the risk or likelihood of a patient developing Type II diabetes and optionally perform one or more intervening actions to reduce this risk or likelihood. For example, the control unit 102 can output one or more signals to an intervention system 110. The signals can indicate the risk or likelihood of a patient developing Type II diabetes. The intervention system 110 can represent one or more automated computerized systems that can receive the risk or likelihood and automatically perform one or more actions, such as scheduling a visit for the patient with a healthcare provider (in-person or via a telehealth appointment), cause a healthcare provider to automatically call or message the patient, schedule food delivery service to aid in improving the diet of the patient, schedule exercise or training sessions to improve the activity level of the patient, schedule mental health counseling sessions with the patient, automatically prescribing (and mailing to the patient) one or more medications, or the like. These actions can be performed to reduce the risk or likelihood of the patient developing Type II diabetes, and can significantly improve the health and extend the lifespan of the patient.

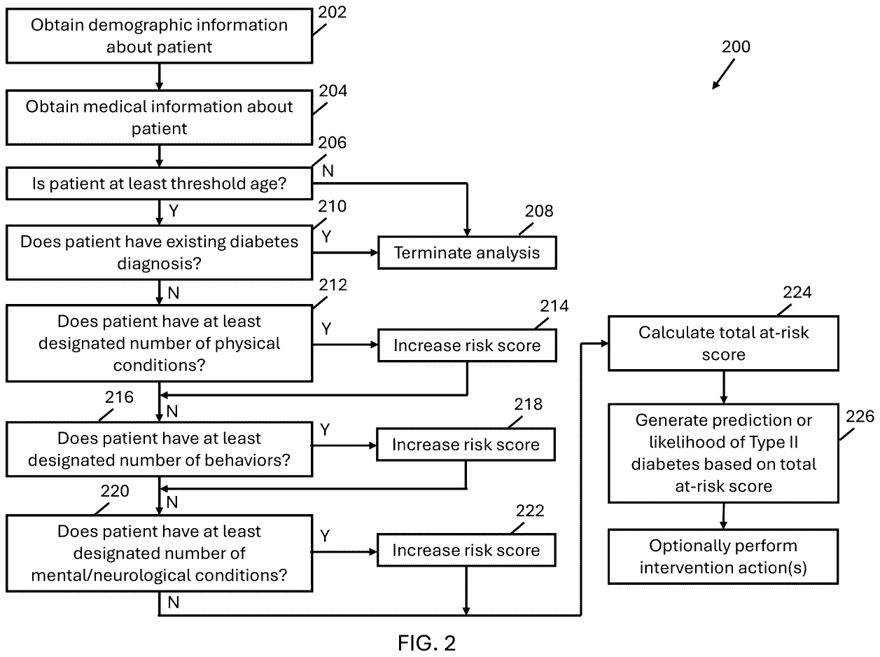

FIG. 2 illustrates a flowchart of a method 200 for predicting a risk or likelihood that a patient will develop Type II diabetes. The method 200 can represent operations performed by the control unit 102 of the prediction system 100 shown in FIG. 1. While all boxes and arrows are drawn in FIG. 2 in solid lines, some operations may be optional and/or performed in a different order, as described herein. The solid lines used to create the boxes and arrows should not be interpreted as a requirement that the operations are all required and/or that the operations are required to be performed in the order shown in FIG. 2 unless otherwise stated herein.

At 202, demographic information about a patient is obtained. This demographic information can include the age of the patient. Optionally, this information can include the gender of the patient, the race of the patient, where the patient resides, and/or where the patient has resided. The demographic information can be received by the memory 106 from one or more of the sources 108, such as by communicating the information via one or more wired and/or wireless computerized communication networks (e.g., the Internet, intranets, virtual private networks, etc.). In another example, the demographic information about the patient is not obtained.

At 204, medical information about the patient is obtained. This medical information can include physical health data or conditions, behavioral data, mental health data, and/or neurological health conditions, as described above. The medical information optionally can be obtained before the demographic information is obtained or at the same time that the demographic information is obtained. In one example, the demographic information may be derived from or obtained from the medical information (thereby eliminating a separate operation of obtaining the demographic information). The medical information can be received by the memory 106 from one or more of the sources 108, such as by communicating the information via one or more wired and/or wireless computerized communication networks.

At 206, the age of the patient is examined, and a decision is made as to whether the patient is at least of a certain (e.g., threshold) age. For example, the control unit 102 can examine the demographic information and/or the medical information to identify how old the patient is. If the patient is old enough to rule out onset or progression toward Type II diabetes, then flow of the method 200 can proceed toward 208. For example, if the patient is an adult (e.g., at least eighteen years old), then the method 200 can proceed toward 208 and terminate. If the patient is not at least the threshold age, then flow of the method 200 can proceed toward 210. For example, if the patient is an adult, then the method 200 can proceed. Alternatively, the method 200 does not include 206. Instead, the method 200 can examine the patient for risk or likelihood of developing Type II diabetes regardless of the age of the patient (e.g., the method 200 can skip the decision at 206).

At 210, a decision is made as to whether the patient already has been diagnosed with diabetes. For example, the control unit 102 can examine the medical history or medical data of the patient to determine whether the patient has been diagnosed with Type I or Type II diabetes. If such a diagnosis already occurred, then the method 200 can proceed toward 208 and terminate. But if such a diagnosis has not occurred, then the method 200 can proceed toward 212. Alternatively, the order of 206 and 210 may be switched up, 206 and 210 may occur at the same time, or the method 200 may not include 206 but may include 210.

At 212, a decision is made as to whether the patient has at least a designated number of physical conditions. The control unit 102 can examine the physical health data received from one or more of the sources 108 to determine whether the physical health data indicates the patient has physical risk factors indicative of a health trend of the patient toward Type II diabetes. For example, the physical health data can be examined to determine whether the patient is obese (e.g., heavier than a designated weight associated with being obese for the patient's height and gender), sedentary (e.g., the patient is less active than a designated level of activity on a daily, weekly, monthly, or annual basis), has high blood pressure (e.g., higher blood pressure than a designated blood pressure for the patient's age, gender, and/or race), is a smoker, has one or more family members (e.g., single generation, multi-generation, or a combination thereof) with a diagnosis of Type II diabetes, and/or has one or more family members (e.g., single generation, multi-generation, or a combination thereof) with a diagnosis of Type I diabetes. These factors can be referred to as physical health factors. If the physical health data indicates that the patient (or family, as appropriate) has at least a threshold number of these physical health factors, then these factors can increase the risk or likelihood of the patient developing Type II diabetes. For example, if the patient has at least a threshold number of these physical health factors, then the patient may be at increased risk or likelihood for developing Type II diabetes (relative to another patient having less than the threshold number of these factors). As a result, flow of the method 200 can proceed toward 214. Otherwise, the patient may not be at increased risk or likelihood for developing Type II diabetes (relative to another patient having less than the threshold number of these factors). As a result, flow of the method 200 can proceed toward 216. The threshold number of physical health factors may be one or another number (e.g., two, three, four, etc.), as determined by using a default value or a value determined by the artificial intelligence or machine learning aspects of the control unit 102 (described below).

At 214, a risk or likelihood score for the patient is increased. For each physical health factor that the patient (or family, as appropriate) has, a risk score may be increased by a default value. This default value can be one or another value. For example, a patient that is obese, is a smoker, and is sedentary may have a risk score of three. Another patient having a family history of diabetes and has high blood pressure may have a risk score of two. The values added to the risk score based on the physical health factors may be weighted more heavily than the values added to the risk score for other health factors described herein. For example, each value added to the risk score due to a physical health factor being present may be doubled (e.g., has a weight of two). Alternatively, another weight may be used (such as three, two and a half, or the like). The weight that is applied to the values added to risk score for the physical health factors may be a default value or may be determined by the artificial intelligence or machine learning aspects of the control unit 102 (described below). Flow of the method 200 can proceed toward 216.

At 216, a decision is made as to whether the patient has at least a designated number of behaviors, or behavioral conditions. The control unit 102 can examine the behavioral health data received from one or more of the sources 108 to determine whether the behavioral health data indicates the patient has behavioral risk factors indicative of a health trend of the patient toward Type II diabetes. For example, the behavioral health data can be examined to determine whether the patient consumes alcohol, has or suffers from obstructive sleep apnea, or has an unhealthy diet. The unhealthy diet may be identified responsive to the patient consuming a low-fiber diet (e.g., consumes less than a designated threshold of fiber on a daily, weekly, or monthly basis) with a high glycemic index (e.g., consumes food having a glycemic index that exceeds a designated glycemic index threshold on a daily, weekly, or monthly basis), consumes processed meats but not consume other, non-processed meats (e.g., more than a designated amount on a daily, weekly, or monthly basis), and/or consumes soda or soft drinks (e.g., consumes more than a threshold amount on a daily, weekly, or monthly basis). These factors can be referred to as behavioral health factors.

As another example, the control unit 102 can examine the demographic data or information about the patient to determine whether the demographic data or information indicates the patient has behavioral risk factors associated with a health trend of the patient toward Type II diabetes. For example, certain states, counties, cities, or the like, may be associated with increased alcohol consumption (e.g., by one or more clinical studies, by WISEVOTER.COM, etc.). If the patient resides in one or more of these states, counties, cities, or the like, then the patient may be found to have a behavioral health factor that increases the risk or likelihood of a trend toward Type II diabetes.

If the behavioral health data indicates that the patient has at least a threshold number of these behavioral health factors, then these factors can increase the risk or likelihood of the patient developing Type II diabetes. For example, if the patient has at least a threshold number of these behavioral health factors, then the patient may be at increased risk or likelihood for developing Type II diabetes (relative to another patient having less than the threshold number of these factors). As a result, flow of the method 200 can proceed toward 218. Otherwise, the patient may not be at increased risk or likelihood for developing Type II diabetes (relative to another patient having less than the threshold number of these factors). As a result, flow of the method 200 can proceed toward 220. The threshold number of behavioral health factors may be one or another number (e.g., two, three, four, etc.), as determined by using a default value or a value determined by the artificial intelligence or machine learning aspects of the control unit 102 (described below).

At 218, a risk or likelihood score for the patient is increased. For each behavioral health factor that the patient has, a risk score may be increased by a default value. This default value can be one or another value. For example, a patient that consumes alcohol may have a risk score of one. Another patient that consumes alcohol and that eats an unhealthy diet (e.g., low-fiber diet with high glycemic index) may have a risk score of two. The values added to the risk score based on the behavioral health factors may be weighted more heavily than the values added to the risk score for other health factors described herein. For example, each value added to the risk score due to a behavioral health factor being present may be doubled (e.g., has a weight of two). Alternatively, another weight may be used (such as three, two and a half, or the like). The weight that is applied to the values added to risk score for the behavioral health factors may be a default value or may be determined by the artificial intelligence or machine learning aspects of the control unit 102 (described below). The addition to the risk score due to the behavioral health factor(s) may be in addition to the values added to the risk score due to other factors, including the physical health factors. Flow of the method 200 can proceed toward 220.

At 220, a decision is made as to whether the patient has at least a designated number of mental or neurological conditions. The control unit 102 can examine the mental health data and/or the neurological health data received from one or more of the sources 108 to determine whether the data indicates the patient has mental risk factors and/or neurological risk factors indicative of a health trend of the patient toward Type II diabetes. For example, the data can be examined to determine whether the patient experienced childhood abuse, whether the patient is a white Caucasian that experienced childhood abuse, whether the patient has a spinal cord injury, whether the patient experienced one or more traumatic events or suffers from post-traumatic stress disorder, whether the patient experiences or is experiencing excess stress, whether the patient has inflammatory processes, and/or whether the patient suffers from depression. These factors can be referred to as mental/neurological health factors, mental health factors, or neurological health factors.

If the mental health data and/or neurological health data indicates that the patient has at least a threshold number of these mental/neurological health factors, then these factors can increase the risk or likelihood of the patient developing Type II diabetes. For example, if the patient has at least a threshold number of these health factors, then the patient may be at increased risk or likelihood for developing Type II diabetes (relative to another patient having less than the threshold number of these factors). As a result, flow of the method 200 can proceed toward 222. Otherwise, the patient may not be at increased risk or likelihood for developing Type II diabetes (relative to another patient having less than the threshold number of these factors). As a result, flow of the method 200 can proceed toward 224. The threshold number of health factors may be one or another number (e.g., two, three, four, etc.), as determined by using a default value or a value determined by the artificial intelligence or machine learning aspects of the control unit 102 (described below).

At 222, a risk or likelihood score for the patient is increased. For each mental/neurological health factor that the patient has, a risk score may be increased by a default value. This default value can be one or another value. For example, a patient that has a spinal cord injury and suffers from depression may have a risk score of two. Another patient that suffers from post-traumatic stress disorder and was abused as a child may have a risk score of two. The values added to the risk score based on the mental/neurological health factors may be weighted less heavily than the values added to the risk score for other health factors described herein. For example, each value added to the risk score due to a mental/neurological health factor being present may not be doubled, but may have a weight of one. Alternatively, another weight may be used (such as one half, three quarters, or the like). The weight that is applied to the values added to risk score for the mental/neurological health factors may be a default value or may be determined by the artificial intelligence or machine learning aspects of the control unit 102 (described below). The addition to the risk score due to the mental/neurological health factor(s) may be in addition to the values added to the risk score due to other factors, including the physical health factors and/or the mental health factors. Flow of the method 200 can proceed toward 224.

At 224, a total at-risk score is calculated. This score may be calculated by adding the physical health factors, behavioral health factors, and mental/neurological health factors that were calculated at 214, 218, 222. Optionally, if the values to the risk score were already added at 214, 218, 222, then 224 may be skipped. The risk score may include the weighted values from these factors, as described above. As one example, the values added to the risk score from the physical health factors and the behavioral health factors may be weighted more heavily in calculating the risk score than the mental/neurological health factors (e.g., double the weight).

At 226, a prediction or likelihood of the patient developing Type II diabetes is made based on the total at-risk score calculated at 224. The control unit 102 can compare the at-risk score with one or more thresholds associated with different likelihoods of the patient developing Type II diabetes. For example, the at-risk score can be compared to a single threshold score value and, if the at-risk score exceeds this threshold, the control unit 102 can predict that the patient will develop Type II diabetes (unless some changes are made, as described herein). As another example, the at-risk score can be compared to different threshold score values associated with different likelihoods of developing Type II diabetes (e.g., <50%, 50-75%, 75-90%, >90%, etc.). The likelihood can be determined based on which of these thresholds the at-risk score exceeds. In another example, the at-risk score can itself be the likelihood of developing Type II diabetes. The at-risk score can be normalized or scaled to a value between 0 and 1, between 0 and 100, etc., with larger values being associated with greater likelihoods of developing Type II diabetes (e.g., a score of 58 indicates a 58% likelihood of developing Type II diabetes).

The risk or likelihood that is determined can be reported to the healthcare provider or patient. Optionally, at 228, one or more intervention or intervening actions can be performed or initiated. For example, the intervention system 110 can automatically schedule a visit for the patient with a healthcare provider (in-person or via a telehealth appointment), cause a healthcare provider to automatically call or message the patient, schedule food delivery service to aid in improving the diet of the patient, schedule exercise or training sessions to improve the activity level of the patient, schedule mental health counseling sessions with the patient, or the like. These actions can be performed to reduce the risk or likelihood of the patient developing Type II diabetes, and can significantly improve the health and extend the lifespan of the patient.

The predictive model 104 used to determine the likelihood that a patient will develop Type II diabetes can be useful in the healthcare and health insurance fields to help healthcare personnel determine whether patients are likely to become diabetic. This early warning will enable a runway to establish clinically proven healthy habits including physical, mental, and behavioral changes, which can prevent the development of diabetes altogether.

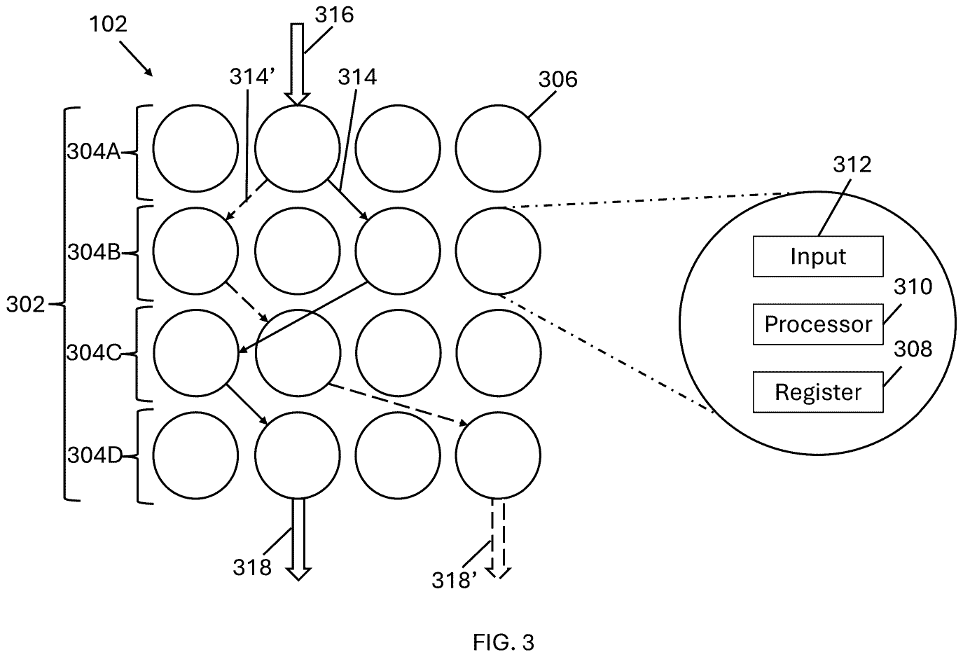

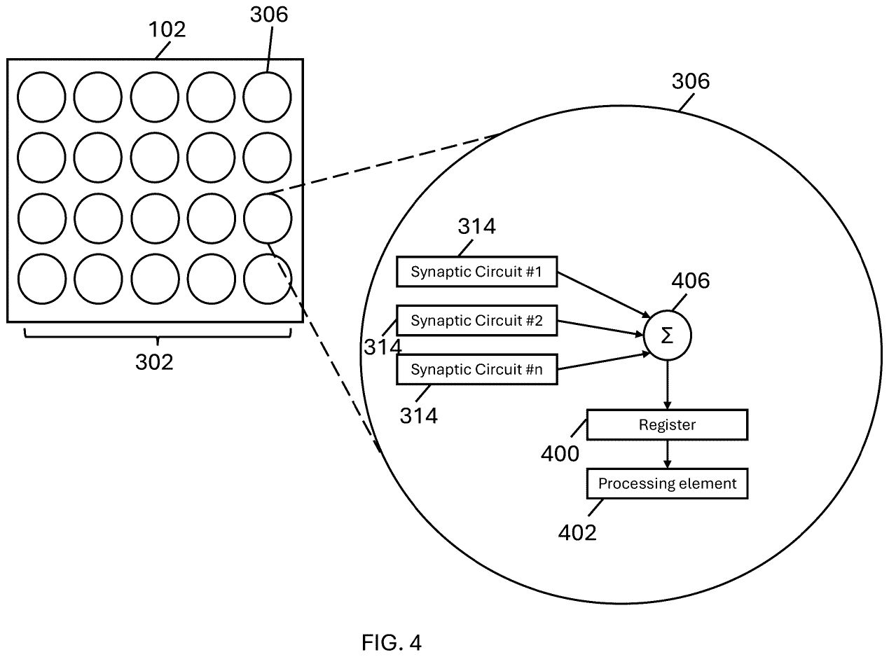

FIG. 3 illustrates one example of an ML/AI system 300. The ML/AI system 300 also can be referred to as an artificial neural network (ANN), and can represent the control unit 102 and the prediction model 104 shown in FIG. 1. The ML/AI system 300 can be embodied in one or more application-specific integrated circuits (ASICs) for an artificial neural network (ANN). With continued reference to the ML/AI system 300 shown in FIG. 3, FIG. 4 illustrates one example of the control unit 102 embodied in at least one ASIC. For example, the control unit 102 can be an ASIC for an ANN, with the control unit 102 including a plurality of neurons 306 organized in an array 302. Each neuron 306 includes a register 400 that is one or more digital storage elements (e.g., latches and/or flip-flops) configured to hold input activations, weights, bias, partial sums, thresholds, parameters, counters, and/or output values associated with the neuron 306 for at least one clock cycle of the AISC. Each neuron 306 also includes a processing element 402, such as circuitry including one or more arithmetic units that compute a weighted combination of neuron inputs and to apply a non-linear activation and associated scaling, biasing, quantization, and/or thresholding to produce output from the neuron 306. The processing element 402 may include multipliers, adders, accumulator registers, shifters, lookup-table logic, comparators, and control logic, and may be implemented in scalar, vector, bit-serial, or analog/mixed-signal form. The neuron 306 also can include at least one input, such as one or more numeric values (e.g., scalar or vector) provided to the processing element 402 of the neuron 306. This input can include activations, residual or recurrent values, or event signals, which can be encoded in digital or analog form and consumed, together with coefficients, to compute the output of the neuron 306. Each neuron 306 also can include a plurality of synaptic circuits 314, such as two or more circuits each receiving a respective input signal and generating a weighted contribution based on a stored synaptic parameter. These contributions are combined at a summing node or accumulator 406 of the neuron 306. Each synaptic circuit 404 may include one or more storage elements for the parameter, switching elements, scaling or multiplication elements, optional delay/decay elements, and gating or masking logic.

Returning to the description of the ML/AI system 300 in FIG. 3, the ASIC(s) can includes the array or series 302 of layers 304A-D, each comprising one or more of the artificial neurons 306 arranged in one or more neuron arrays or arrangements. While four neurons 306 are shown in each layer 304A-D and four layers 304A-D are shown, alternatively, a different number of neurons 306 may be in one or more of the layers 304A-D and/or there may be a different number of layers 304A-D.

The ML/AI system 300 may include the neurons 306 arranged in an input layer 304A, an output layer 304D, and two or more fully connected hidden or intermediate layers 304B, 304C between the input and output layers 304A, 304D. Each neuron 306 can include or represent a register 308, a microprocessor 310, and at least one input 312. The neurons 306 can generate output based on one or more activation functions. The neurons 306 can receive input from another neuron 306 (e.g., the output from one neuron 306 can be the input for another neuron 306). This input also can include a set of weights. The neurons 306 can be connected with each other via synaptic circuits 314, 314′. The synaptic circuits 314, 314′ can include or represent memories for storing synaptic weights.

One or more neurons 306 in the input layer 304A of the ML/AI system 300 can receive an input 316 into the ML/AI system 300. The input 316 can include, for example, one or more of the demographic data, the physical health data, the mental health data, the neurological health data, and/or the behavioral health data. The neurons 306 can receive this input data 316 via the input(s) 312 of the neurons 306 in the input layer 304A. The neurons 306 receive the input data 316, apply one or more mathematical equations or relationships stored in the registers 308 (and that include the weights) to generate an output. The processors 310 of the neurons 306 apply the equations/relationships and can pass the output to another neuron 306 in the same layer 304A or in a different layer 304B, 304C. The output from one neuron 306 is passed along a synaptic circuit 314 to another neuron 306 and is used as input to this other neuron 306. This process continues until one or more neurons 306 in the output layer 304D generate an output 318 from the ML/AI system 300.

The synaptic circuits 314, 314′, weights stored in the synaptic circuits 314, 314′, and/or the mathematical relationships between the neurons 306 can define the prediction model 104. For example, the AI/ML system 300 can examine the input data 316 and identify the risk factors present (if any) in the different categories of health data, compare the identified risk factors to the appropriate thresholds, determine whether to increase or add to the at-risk score based on this comparison, apply the weights (if any) to the values added to the at-risk score, and determine the risk or likelihood of the patient developing Type II diabetes, as described above. The weights, values, and likelihoods can define the synaptic circuits 314, 314′, weights stored in the synaptic circuits 314, 314′, and/or the mathematical relationships between the neurons 306.

During training of the AI/ML system 300, labeled data may be provided as the input data 316 to the AI/ML system 300. The labeled data can include health data of other patients known to have eventually been diagnosed with Type II diabetes, other patients known to have eventually been diagnosed with Type II diabetes and then Type I diabetes, and/or other patients known to have not been diagnosed with Type II diabetes. The neurons 306 process the input data 316 to generate the training output of the AI/ML system 300. This training output can identify likelihoods that these other patients would have developed Type II (and/or Type I) diabetes.

Feedback can be provided to the AI/ML system 300 in the form of a calculated error or other indication of the differences between the likelihood of developing Type II diabetes output by the system AI/ML system 300 and the actual patient development (or non-development) of Type II (and/or Type I) diabetes. For example, if the AI/ML system 300 identified a high likelihood that a patient associated with the training health data was going to develop Type II diabetes, but the patient never developed Type II diabetes, then a larger error is calculated. If the AI/ML system 300 identified a high likelihood that a patient associated with the training health data was going to develop Type II diabetes, and the patient developed Type II diabetes, then a smaller error (or no error) is calculated. If the AI/ML system 300 identified a low likelihood that a patient associated with the training health data was going to develop Type II diabetes, and the patient developed Type II diabetes, then a larger error is calculated. If the AI/ML system 300 identified a low likelihood that a patient associated with the training health data was going to develop Type II diabetes, and the patient never developed Type II diabetes, then a smaller error (or no error) is calculated.

Based on this error, the neurons 306 can change one or more of the synaptic circuits 314 that connect the neurons 306, the weights applied by one or more of the neurons 306, and/or the mathematical relationships between the neurons 306. For example, some synaptic circuits 314 can be changed to modified synaptic circuits 314′ such that the same input 316 would result in different neurons 306 receiving input and passing output to other neurons and generating a different output 318′ from the AI/ML system 300. As a result, changing one or more of these weights or relationships (e.g., synaptic circuits 314, 314′) also can change the at-risk score, one or more of the weights applied to the values added to the at-risk score, the thresholds used to determine whether to increase the at-risk score, the threshold(s) to which the at-risk score is compared to predict the likelihood of developing Type II diabetes, etc.

After training the AI/ML system 300, the AI/ML system 300 can use the trained model(s) to predict the likelihood for patients to develop Type II diabetes. During post-training iterations of operation of the AI/ML system 300, additional feedback can be provided to the AI/ML system 300 based on errors in the predicted likelihood. For example, after training, progress of the patients toward (or away from) developing Type II diabetes can be examined and used to generate additional feedback for the AI/ML system 300. The AI/ML system 300 can repeatedly receive such feedback, modify one or more weights and/or synaptic circuits, etc. so that the AI/ML system 300 repeatedly changes to improve and reduce error in predicting the risk or likelihood of patients developing Type II diabetes.

The subject matter described and claimed herein is integrated into a practical application that improves the functioning of a computer-implemented predictive system and applies that improvement to a concrete medical workflow. For example, the control unit obtains multiple categories of non-conventional health data from disparate sources (e.g., behavioral health, mental health, and neurological health data in addition to physical health data), to identify and weigh risk factors differently across those categories in a trained model, and to output signals that cause specified intervention actions when a designated threshold is met. The control unit may include or operate in connection with an ASIC that includes neurons, synaptic circuits, and memories storing synaptic weights, where the processing elements of the neurons perform the recited data acquisition, risk identification, score calculation, and initiation of interventions. These aspects go beyond a mere mental process or a generic instruction to “apply it.” Rather, these aspects tie the subject matter described herein to a particular machine architecture and to an improvement in how such machine processes heterogeneous health data with specific weighting schemes to generate low-latency, on-device predictions that trigger concrete medical interventions.

Similar to patent-eligible technologies that apply a natural relationship in a specific treatment protocol, the method(s) described herein can require usage of a trained model that differentially weighs heterogeneous categories of health data and conditionally initiates particular intervention actions (e.g., scheduling a provider visit, arranging food delivery, or scheduling exercise and mental health counseling) based on a designated threshold. This is not merely reporting a risk. Instead, this methodology is a specific, rule-based medical workflow that changes the course of care for a particular patient population before the onset of Type II diabetes. Likewise, the subject matter described herein does not preempt the field of diabetes risk assessment. Instead, the subject matter provides a concrete implementation that requires obtaining and processing a defined combination of whole-person-health criteria, applying a particular weighting relationship across categories, and initiating defined follow-on actions through an intervention system.

Usage of an ASIC, in particular, provides a non-generic hardware configuration with neurons organized in arrays, synaptic circuits with memories storing synaptic weights, and processing elements configured to implement the specific data acquisition, identification, and weighted-scoring operations recited, all in hardware. This architecture is not a generic computer performing routine functions, but reflects an unconventional arrangement that yields concrete benefits such as reduced inference latency, energy-efficient on-chip scoring, and improved privacy by eliminating the need to transmit protected health information to remote servers. Similarly, the systems provide a control unit that obtains and processes specified categories of data, apply differential weights across those categories to produce an at-risk score, and generate signals to an intervention system that automatically executes enumerated actions. This recited combination of specific data pipelines, weighting regimen, thresholding, and machine-initiated interventions constitutes significantly more than a mathematical calculation or mere data display.

The subject matter described herein cannot be performed mentally or with pen and paper. The subject matter described herein can involve obtaining and processing multiple categories of patient and population-level health data (including data from sensors and provider systems), identifying and applying different weights to multiple risk factors across categories, and performing these operations using an ANN implemented in ASIC circuitry with synaptic memories that store the trained weights. The scale, data heterogeneity, and specific hardware execution recited cannot be practically performed by the human mind, as the human mind is not equipped to perform these executed operations within a realistic or reasonable time frame (e.g., before the time for intervention has passed and the patient develops Type II diabetes, kidney disease, etc.).

Additionally, the subject matter described herein is integrated into a practical application, such as: (i) the intervention actions being automatically initiated by the control unit or ASIC upon determining that the at-risk score exceeds the designated threshold; (ii) the model's weighting scheme is trained and stored in synaptic memories of the ASIC and quantized for on-chip inference; and/or (iii) the particular data sources and non-conventional combination of behavioral, mental, neurological, and physical data used in computing the at-risk score.

In one example, a method for predicting a likelihood of a patient developing Type II diabetes is provided. The method can include obtaining one or more of behavioral health data, mental health data, or neurological health data of the patient, identifying one or more risk factors from the one or more of the behavioral health data, the mental health data, or the neurological health data, and calculating an at-risk score based on the one or more risk factors, the at-risk score indicative of the likelihood of the patient developing Type II diabetes. The method also can include implementing one or more intervention actions responsive to the at-risk score exceeding a designated threshold. The one or more intervention actions can include one or more of scheduling a visit with a healthcare provider, delivering meals to the patient, scheduling exercise or training sessions with the patient, scheduling mental health counseling sessions with the patient, or prescribing one or more medications to the patient. The behavioral health data can be obtained and includes one or more of the patient consuming alcohol, the patient having obstructive sleep apnea, the patient consuming a low-fiber diet with a high glycemic index, the patient consuming processed meats and insufficient non-processed meats, or the patient consuming soda. The mental health data can be obtained and include one or more of the patient experiencing childhood abuse, the patient having post-traumatic stress disorder, the patient experiencing a traumatic event, the patient having excess stress, or the patient having a depression diagnosis. The neurological health data can be obtained and include one or more of the patient having a spinal cord injury, or the patient having an inflammatory process. The method also can include obtaining physical health data, and the one or more risk factors also can be identified from the physical health data. The physical health data can include one or more of a family history of diabetes, the patient being obese, the patient having a sedentary lifestyle, the patient having high blood pressure, or the patient being a smoker. The at-risk score can be calculated based on multiple ones of the one or more risk factors. The at-risk score can be calculated by weighing the multiple ones of the one or more risk factors differently. The risk factors from the behavioral health data can be weighed more heavily than the risk factors from the mental health data or from the neurological health data in calculating the at-risk score. The risk factors from the physical health data can be weighed more heavily than the risk factors from the mental health data or from the neurological health data in calculating the at-risk score.

In another example, an ASIC for an ANN is provided. The ASIC can include neurons organized in an array, with each of the neurons including a register, a processing element, and at least one input. The ASIC also can include synaptic circuits, with each of the synaptic circuits including a memory for storing a synaptic weight. Each of the neurons can be connected to at least one other of the neurons via at least one of the synaptic circuits. The processing elements of the neurons can obtain one or more of behavioral health data, mental health data, or neurological health data of a patient, identify one or more risk factors from the one or more of the behavioral health data, the mental health data, or the neurological health data, and calculate an at-risk score based on the one or more risk factors, the at-risk score indicative of a likelihood of the patient developing Type II diabetes. The processing elements of the neurons can implement one or more intervention actions responsive to the at-risk score exceeding a designated threshold. The one or more intervention actions can include one or more of scheduling a visit with a healthcare provider, delivering meals to the patient, scheduling exercise or training sessions with the patient, scheduling mental health counseling sessions with the patient, or prescribing one or more medications to the patient. The processing elements of the neurons can obtain the behavioral health data that includes one or more of the patient consuming alcohol, the patient having obstructive sleep apnea, the patient consuming a low-fiber diet with a high glycemic index, the patient consuming processed meats and insufficient non-processed meats, or the patient consuming soda. The processing elements of the neurons can obtain the mental health data that includes one or more of the patient experiencing childhood abuse, the patient having post-traumatic stress disorder, the patient experiencing a traumatic event, the patient having excess stress, or the patient having a depression diagnosis. The processing elements of the neurons can obtain the neurological health data that includes one or more of the patient having a spinal cord injury, or the patient having an inflammatory process.

In another example, a Type II diabetes prediction system is provided. The prediction system can include a control unit that can obtain one or more of behavioral health data, mental health data, or neurological health data of the patient. The control unit can identify one or more risk factors from the one or more of the behavioral health data, the mental health data, or the neurological health data. The control unit can calculate an at-risk score based on the one or more risk factors. The at-risk score can indicate the likelihood of the patient developing Type II diabetes. The control unit can output the likelihood of the patient developing Type II diabetes to an intervention system that implements one or more intervention actions responsive to the at-risk score exceeding a designated threshold. The one or more intervention actions can include one or more of scheduling a visit with a healthcare provider, delivering meals to the patient, scheduling exercise or training sessions with the patient, scheduling mental health counseling sessions with the patient, or prescribing one or more medications to the patient. The control unit can obtain the behavioral health data that includes one or more of the patient consuming alcohol, the patient having obstructive sleep apnea, the patient consuming a low-fiber diet with a high glycemic index, the patient consuming processed meats and insufficient non-processed meats, or the patient consuming soda. The control unit can obtain the mental health data that includes one or more of the patient experiencing childhood abuse, the patient having post-traumatic stress disorder, the patient experiencing a traumatic event, the patient having excess stress, or the patient having a depression diagnosis. The control unit can obtain physical health data and identify the one or more risk factors using the physical health data. The control unit can calculate the at-risk score based on multiple ones of the one or more risk factors. The control unit can calculate the at-risk score by weighing the multiple ones of the one or more risk factors differently. The risk factors from the behavioral health data can be weighed more heavily than the risk factors from the mental health data or from the neurological health data in calculating the at-risk score. The risk factors from the physical health data can be weighed more heavily than the risk factors from the mental health data or from the neurological health data in calculating the at-risk score.

As used herein, a structure, limitation, or element that is “configured to” perform a task or operation is particularly structurally formed, constructed, or adapted in a manner corresponding to the task or operation. For purposes of clarity and the avoidance of doubt, an object that is merely capable of being modified to perform the task or operation is not “configured to” perform the task or operation as used herein.

It is to be understood that the above description is intended to be illustrative, and not restrictive. For example, the above-described examples (and/or aspects thereof) can be used in combination with each other. In addition, many modifications can be made to adapt a particular situation or material to the teachings of the various examples of the disclosure without departing from their scope. For example, the embodiments of the present application may be combined with or modified to include the systems and methods described in U.S. Pat. Nos. 8,112,288; 10,192,034; and 11,688,513 and U.S. Patent Publication No. 2023-0093336, which are all hereby incorporated by reference. While the dimensions and types of materials described herein are intended to define the aspects of the various examples of the disclosure, the examples are by no means limiting and are exemplary examples. Many other examples will be apparent to those of skill in the art upon reviewing the above description. The scope of the various examples of the disclosure should, therefore, be determined with reference to the appended claims, along with the full scope of equivalents to which such claims are entitled. In the appended claims and the detailed description herein, the terms “including” and “in which” are used as the plain-English equivalents of the respective terms “comprising” and “wherein.” Moreover, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112(f), unless and until such claim limitations expressly use the phrase “means for” followed by a statement of function void of further structure.

This written description uses examples to disclose the various examples of the disclosure, including the best mode, and also to enable any person skilled in the art to practice the various examples of the disclosure, including making and using any devices or systems and performing any incorporated methods. The patentable scope of the various examples of the disclosure is defined by the claims, and can include other examples that occur to those skilled in the art. Such other examples are intended to be within the scope of the claims if the examples have structural elements that do not differ from the literal language of the claims, or if the examples include equivalent structural elements with insubstantial differences from the literal language of the claims.

What is claimed is:

1. An application-specific integrated circuit (ASIC) for an artificial neural network (ANN), the ASIC comprising:
neurons organized in an array, each of the neurons including a register, a processing element, and at least one input; and
synaptic circuits, each of the synaptic circuits including a memory for storing a synaptic weight, wherein each of the neurons is connected to at least one other of the neurons via at least one of the synaptic circuits, the processing elements of the neurons configured to:
obtain one or more of behavioral health data, mental health data, or neurological health data of a patient;
identify one or more risk factors from the one or more of the behavioral health data, the mental health data, or the neurological health data; and
calculate an at-risk score based on the one or more risk factors, the at-risk score indicative of a likelihood of the patient developing Type II diabetes.

2. The ASIC of claim 1, wherein the processing elements of the neurons are configured to implement one or more intervention actions responsive to the at-risk score exceeding a designated threshold, the one or more intervention actions including one or more of:
scheduling a visit with a healthcare provider;
delivering meals to the patient;
scheduling exercise or training sessions with the patient;
scheduling mental health counseling sessions with the patient; or
prescribing one or more medications to the patient.

3. The ASIC of claim 1, wherein the processing elements of the neurons are configured to obtain the behavioral health data that includes one or more of:
the patient consuming alcohol;
the patient having obstructive sleep apnea;
the patient consuming a low-fiber diet with a high glycemic index;
the patient consuming processed meats and insufficient non-processed meats; or
the patient consuming soda.

4. The ASIC of claim 1, wherein the processing elements of the neurons are configured to obtain the mental health data that includes one or more of:
the patient experiencing childhood abuse;
the patient having post-traumatic stress disorder;
the patient experiencing a traumatic event;
the patient having excess stress; or
the patient having a depression diagnosis.

5. The ASIC of claim 1, wherein the processing elements of the neurons are configured to obtain the neurological health data that includes one or more of:
the patient having a spinal cord injury; or
the patient having an inflammatory process.

6. A Type II diabetes prediction system, the prediction system comprising:
an application-specific integrated circuit (ASIC) having neurons organized in an array, each of the neurons including a register, a processing element, and at least one input, the ASIC also including synaptic circuits with each of the synaptic circuits including a memory for storing a synaptic weight, each of the neurons is connected to at least one other of the neurons via at least one of the synaptic circuits, the processing elements of the neurons configured to obtain one or more of behavioral health data, mental health data, or neurological health data of a patient, the control unit configured to identify one or more risk factors from the one or more of the behavioral health data, the mental health data, or the neurological health data, the control unit configured to calculate an at-risk score based on the one or more risk factors, the at-risk score indicative of a likelihood of the patient developing Type II diabetes.

7. The prediction system of claim 6, wherein the ASIC is configured to output the likelihood of the patient developing Type II diabetes to an intervention system that implements one or more intervention actions responsive to the at-risk score exceeding a designated threshold, the one or more intervention actions including one or more of scheduling a visit with a healthcare provider, delivering meals to the patient, scheduling exercise or training sessions with the patient, scheduling mental health counseling sessions with the patient, or prescribing one or more medications to the patient.

8. The prediction system of claim 6, wherein the ASIC is configured to obtain the behavioral health data that includes one or more of the patient consuming alcohol, the patient having obstructive sleep apnea, the patient consuming a low-fiber diet with a high glycemic index, the patient consuming processed meats and insufficient non-processed meats, or the patient consuming soda.

9. The prediction system of claim 6, wherein the ASIC is configured to obtain the mental health data that includes one or more of the patient experiencing childhood abuse, the patient having post-traumatic stress disorder, the patient experiencing a traumatic event, the patient having excess stress, or the patient having a depression diagnosis.

10. The prediction system of claim 6, wherein the ASIC is configured to obtain physical health data and identify the one or more risk factors using the physical health data, the ASIC configured to calculate the at-risk score based on multiple ones of the one or more risk factors, the ASIC configured to calculate the at-risk score by weighing the multiple ones of the one or more risk factors differently, wherein the risk factors from the behavioral health data are weighed more heavily than the risk factors from the mental health data or from the neurological health data in calculating the at-risk score, and the risk factors from the physical health data are weighed more heavily than the risk factors from the mental health data or from the neurological health data in calculating the at-risk score.

11. A method for predicting a likelihood of a patient developing Type II diabetes, the method comprising:
obtaining one or more of behavioral health data, mental health data, or neurological health data of the patient;
identifying one or more risk factors from the one or more of the behavioral health data, the mental health data, or the neurological health data; and
calculating an at-risk score based on the one or more risk factors, the at-risk score indicative of the likelihood of the patient developing Type II diabetes.

12. The method of claim 11, further comprising:
implementing one or more intervention actions responsive to the at-risk score exceeding a designated threshold, the one or more intervention actions including one or more of:
scheduling a visit with a healthcare provider;
delivering meals to the patient;
scheduling exercise or training sessions with the patient;
scheduling mental health counseling sessions with the patient; or
prescribing one or more medications to the patient.

13. The method of claim 11, wherein the behavioral health data is obtained and includes one or more of:
the patient consuming alcohol;
the patient having obstructive sleep apnea;
the patient consuming a low-fiber diet with a high glycemic index;
the patient consuming processed meats and insufficient non-processed meats; or
the patient consuming soda.

14. The method of claim 11, wherein the mental health data is obtained and includes one or more of:
the patient experiencing childhood abuse;
the patient having post-traumatic stress disorder;
the patient experiencing a traumatic event;
the patient having excess stress; or
the patient having a depression diagnosis.

15. The method of claim 11, wherein the neurological health data is obtained and includes one or more of:
the patient having a spinal cord injury; or
the patient having an inflammatory process.

16. The method of claim 11, further comprising:
obtaining physical health data, wherein the one or more risk factors also are identified from the physical health data.

17. The method of claim 16, wherein the physical health data includes one or more of:
a family history of diabetes;
the patient being obese;
the patient having a sedentary lifestyle;
the patient having high blood pressure; or
the patient being a smoker.

18. The method of claim 16, wherein the at-risk score is calculated based on multiple ones of the one or more risk factors, the at-risk score calculating by weighing the multiple ones of the one or more risk factors differently.

19. The method of claim 18, wherein the risk factors from the behavioral health data are weighed more heavily than the risk factors from the mental health data or from the neurological health data in calculating the at-risk score.

20. The method of claim 19, wherein the risk factors from the physical health data are weighed more heavily than the risk factors from the mental health data or from the neurological health data in calculating the at-risk score.
